(19) United States
(12) Patent Application Publication
(10) Pub. No.: US 2019/0336436 A1
(43) Pub. Date: Nov. 7, 2019

(54) NEW USE OF VINEYARD TEARS FOR THE PREPARATION OF COSMETIC COMPOSITIONS

(71) Applicant: Gabriela HAIAS, Perret (FR)

(72) Inventor: Gabriela HAIAS, Perret (FR)

(21) Appl. No.: 16/330,294

(22) PCT Filed: Oct. 3, 2017
(86) PCT No.: PCT/FR2017/000182

§ 371 Date: Jul. 16, 2019

(30) Foreign Application Priority Data
Oct. 6, 2016 (FR) 16/01453

(51) Int. Cl. A61K 8/9789; A61K 8/98; A61Q 19/08

(52) U.S. Cl. CPC A61K 8/9789; A61Q 19/08; A61K 8/986; A61K 8/987

(57) ABSTRACT

As disclosed, vineyard tears in which a set of flowers or plants or algae was initially dipped and, a vegetable oil in which the set of flowers or plants or algae was immersed after having been soaked in the vineyard tears, wherein the set of flowers or plants or algae is optionally milled after having been soaked in the vineyard tears or after having been soaked in the vegetable oil, are used for the preparation of a cosmetic composition, the composition being free of grape seed polyphenols.

The present invention relates to a new use of vineyard tears for the preparation of cosmetic compositions.

Throughout the centuries, the popular pharmacopoeia attributed to vineyard tears, also known as “tears of vine”, all sorts of virtues, and vineyard tears are particularly used to treat ocular affections.

The unequaled qualities of flowers such as rose, have led to their use in many cosmetics. Other flowers such as violets are mainly used in perfumery but less for their beneficial effect on the skin.

In cosmetics, vegetable oils have become unavoidable. Rich in unsaturated fatty acids, they penetrate the epidermis well, protect it by reconstituting the lipidic film, give it finesse, brightness and suppleness. They do not present an incompatibility with the body and play a preponderant role in the protection and hydration of our skin.

Many cosmetic creams exist to date but are sometimes inappropriate especially for mature skin, which lack elasticity and tone. Some creams have negative effects on the skin and can cause blisters, redness, itching, or tightness of the skin.

There is therefore a real need to obtain cosmetic compositions that can overcome the aforementioned drawbacks.

The applicant has found, surprisingly, that the combination of vineyard tears with certain flowers or plants makes it possible to improve the properties of the cosmetic compositions. Indeed, the synergistic action of these two elements has anti-wrinkle effects, anti-stain, anti-oxidant, illuminator of complexion, anti-fatigue, stimulates the formation of natural collagen, stimulates the elasticity and the tonicity of the skin.

The object of the invention is to provide a cosmetic composition with improved properties and the means necessary for obtaining this cosmetic composition.

The present invention thus relates to the use of a mixture based on vineyard tears and vegetable oil for the preparation of a cosmetic composition.

The present invention also relates to a cosmetic composition comprising said mixture mentioned above.

In a first aspect, the present invention relates to the use of a mixture comprising:
vineyard tears in which an initial set of flowers or plants or algae was dipped and,

A vegetable oil in which said set of flowers or plants or algae has been immersed after having been soaked in said vineyard tears,

wherein said set of flowers or plants or algae is optionally milled after having been soaked in said vineyard tears or after having been soaked in said vegetable oil,
for the preparation of a cosmetic composition.

“Vineyard tears” mean the substance that flows from the end of pruned vine shoots and that correspond to the resumption of activity of the root system of the vine. Vineyard tears appear once a year, especially in March when the soil temperature is greater than or equal to 10° C. Vineyard tears flow during about 10 days a year.

“Set of flowers or plants” means a lot of the same flowers or a lot of the same plants or a combination of a lot of the same flowers with a lot of the same plants. This set of flowers or plants may consist of all the elements that make up these flowers or plants.

“Vegetable oil” means a fatty substance extracted from an oleaginous plant, meaning a plant whose seeds, nuts or fruits contain lipids.

“Aqueous extract” means the substance obtained following the grinding of said set of flowers or plants after it has soaked in said vineyard tears.

“Oily extract” means the substance obtained following the grinding of said set of flowers or plants after it has soaked in said vegetable oil.

In a particular embodiment, the invention relates to the said aforementioned use and is characterized in that the said cosmetic composition is free of grape seed polyphenols.

In a particular embodiment, the invention relates to the said use in which the flowers or plants of the said set of flowers or plants mentioned above are selected between:

Agrimony (Agrimonia eupatoria); Alchemilla (Alchemilla vulgaris); Green Anise (Pimpinella anisum) Anthyllis (Anthyllis vulneraria); Armenia white grass (Artemisia herba-alba); Arnica (Arnica montana); Hawthorn (Crataegus oxyacantha); Four o-clock flower (Mirabilis jalapa); Cornflower (Centaurea cyanus); Buddleia; Calendula (Calendula officinalis); Camellia (Camellia sinensis); Chamomile (Anthemis nobilis); Centella (Centella asiatica); Echinacea (Echinacea purpurea); True rosehip; Eleutherococcus; Frangipani (Plumeria alba); Passion fruit flowers; Scotch broom (Cytisus scoparius); Juniper (Juniperus communis); Phenicia juniper; Gentian (Gentiana lutea); Geranium rosat; Pelargonium rosat group; Great comfrey (Symphytum officinale); Marshmallow (Althaea officinalis); Hamamelis (Hamamelis virginiana); Hibiscus; Himanthalia, also known as sea spaghetti (Himanthalia elongata); Immortal; Iris (Iris florentina); Himalayan Jasmine (Nyctantes arbor-tristis); Officinal jasmine, also known as common jasmine (Jasminum officinale); Sambac jasmine, also known as jasmine of Arabia; Daffodil; White cutthroat or white nettle (Lamium album); Laurel; Lavandin; White limnanthes, also known as meadowfoam (Limnanthes alba); white or purple lilac; Lotus; White lupine (Lipinus albus); Lily; Marjoram (Origanum majorana); Horse Chestnut (Aesculus hippocastanus); Mallow or Great Mallow (Malva sylvestris); St. John's wort (Hypericum perforatum); Mimosa (Mimosa tenuiflora); Moringa (Moringa oleifera); Lily of the Valley; Narcissus; White water lily (Nymphea alba); Orange tree (Citrus amara) blossom or fruit pulp; Blue orchid (Dendrobium phalaenopsis); White orchid; Palmarosa (Cymbopogon martini); Peach blossom (Prunus persica); Pelargonium with a strong odor (Pelargonium graveolens); Pelargonium with flowering head (Pelargonium capitatum); Apple blossom; Plum blossom; Pelvetia (Pelvetia canaliculata); Heartsease (Viola tricolor); Periwinkle of Madagascar (Vinca rosea); Peony; Psyllium, also known as ispaghula (Plantago ovata); Purslane (Portulaca oleracea); Horsetail (Equisetum arvense); Meadowsweet (Spiraea ulmaria); Rose of France (Rosa gallica); Rose of Damascus; Musky rose; Saffron (Crocus sativus); Officinal saponaria (Saponaria officinalis); Clary Sage flower; Saxifrage (Saxifraga sarmentosa); Scutellaria (Scutellaria baicalensis); Elderberry (Sambucus nigra); Large-leaved lime blossom (Tilia platyphyllos); Small-leaved lime blossom (Tilia cordata); Sunflower (Helianthus annuus); Tuberose (Polianthes tuberosa); Valerian (Valeriana officinalis); Bourbon vanilla blossom; Brazilian vanilla blossom (Vanilla planifolia); Lemon verbena (Aloysia citrodora); Violet; Ylang-ylang (Cananga odorata); Hedera helix; Alchillea millefolium; Officinal symphytum; Populus nigra; Salvia officinalis; Plantago major; Arctium lappa; Nigritella rubra; Prunus spinosa; Seabuckthorn blossom

or a mixture of these flowers,
or the following algae: Ascophyllum nodusum; Laminaria; Fucus vesiculosum; Chondrus crispus; Lithotamnium calcareum; Absidinum helminthocorton; Diginea simplex; sea fennel or a mixture of these algae.

In a particular embodiment, the invention relates to said use wherein said set of flowers comprises or consists of violets or roses, including Damask roses, or a mixture of these flowers.

In another particular embodiment, the invention relates to the said use wherein the said vegetable oil is selected from: sweet almond oil, baobab oil, camelina oil, Inca Inchi oil, jojoba oil, hazelnut oil, calophyll oil, andiroba oil, raspberry seed oil, argan oil, borage oil, lemon oil, hemp oil, prickly pear oil, wheat germ oil, cashew nut oil, strawberry seed oil, rose hip oil, avocado oil, seabuckthorn oil, evening primrose oil, apricot kernel oil, macadamia oil, inophyllous calophyll oil, black cumin oil, neem oil, sesame oil, olive oil, monoi oil, papaya oil, safflower oil, rice bran oil, pumpkin seed oil, bellise oil, babassu oil, calendula oil, camellia oil, pequi oil, pracaxi oil, rosehip oil, sunflower oil or copaiba oil or a mixture of these oils.

In yet another particular embodiment, the invention relates to the said use in which the said vineyard tears comprise the following elements: benzyl alcohol, butanol, butyl acetate, carvacrol, beta-caryophyllenoxide, 1,8-cineol, cuminaldehyde, 1,2-dimethylbenzol, acetic acid, ethyl acetate, beta-famesen, beta-farnesol, hexanol, 1-hexen-3-ol, hexyl acetate, camphor, myristic acid, octanol, palmitic acid, methyl ester, palmitic acid, alpha-pinen, propanol, sabinene, stearic acid, alpha-terpineol, terpinol, terpinyl acetate, tetradecan, thymol, tridecan, 1,2,3-trimethylbenzol, saponin.

In a particular embodiment, the invention relates to the said use in which the weight ratio between the said vineyard tears and the said vegetable oil varies from 0.08 to 16.

This ratio may especially be 0.10; 0.20; 0.30; 0.40; 0.50; 0.6; 0.7; 0.8; 0.9; 1; 1.5; 2; 2.5; 3; 3.5; 4; 4.5; 5; 5.5; 6; 6.5; 7; 7.5; 8; 8.5; 9; 9.5; 10; 10.5; 11; 11.5; 12; 12.5; 13; 13.5; 14; 14.5; 15; 15.5 or 16.

In another particular embodiment, the invention relates to the said use in which the said flowers or plants of the set of flowers or plants can be fresh flowers or fresh plants, dried flowers or dried plants or a mixture of these flowers or plants.

In a second aspect, the present invention relates to a cosmetic composition comprising a mixture of:
vineyard tears in which an initial set of flowers or plants or algae was dipped in and,
a vegetable oil in which the said set of flowers or plants or algae has previously been dipped after having been initially soaked in said vineyard tears wherein the said set of flowers or plants or algae is optionally milled after having been soaked in the said vineyard tears or after having been soaked in the said vegetable oil.

In a particular embodiment the invention relates to the said cosmetic composition characterized in that it is free of grape seed polyphenols.

In another particular embodiment, the invention relates to the said cosmetic composition in which the flowers or plants of the said set of flowers or plants are selected between: Agrimony (Agrimonia eupatoria); Alchemilla (Alchemilla vulgaris); Green Anise (Pimpinella anisum) Anthyllis (Anthyllis vulneraria); Armenia white grass (Artemisia herba-alba); Arnica (Arnica montana); Hawthorn (Crataegus oxyacantha); Four o-clock flower (Mirabilis jalapa); Cornflower (Centaurea cyanus); Buddleia; Calendula (Calendula officinalis); Camellia (Camellia sinensis); Chamomile (Anthemis nobilis); Centella (Centella asiatica); Echinacea (Echinacea purpurea); True rosehip; Eleutherococcus; Frangipani (Plumeria alba); Passion fruit flowers; Scotch broom (Cytisus scoparius); Juniper (Juniperus communis); Phenicia juniper; Gentian (Gentiana lutea); Geranium rosat; Pelargonium rosat group; Great comfrey (Symphytum officinale); Marshmallow (Althaea officinalis); Hamamelis (Hamamelis virginiana); Hibiscus; Himanthalia, also known as sea spaghetti (Himanthalia elongata); Immortal; Iris (Iris florentina); Himalayan Jasmine (Nyctantes arbor-tristis); Officinal jasmine, also known as common jasmine (Jasminum officinale); Sambac jasmine, also known as jasmine of Arabia; Daffodil; White cutthroat or white nettle (Lamium album); Laurel; Lavandin; White limnanthes, also known as meadowfoam (Limnanthes alba); white or purple lilac; Lotus; White lupine (Lipinus albus); Lily; Marjoram (Origanum majorana); Horse Chestnut (Aesculus hippocastanus); Mallow or Great Mallow (Malva sylvestris); St. John's wort (Hypericum perforatum); Mimosa (Mimosa tenuiflora); Moringa (Moringa oleifera); Lily of the Valley; Narcissus; White water lily (Nymphea alba); Orange tree (Citrus amara) blossom or fruit pulp; Blue orchid (Dendrobium phalaenopsis); White orchid; Palmarosa (Cymbopogon martini); Peach blossom (Prunus persica); Pelargonium with a strong odor (Pelargonium graveolens); Pelargonium with flowering head (Pelargonium capitatum); Apple blossom; Plum blossom; Pelvetia (Pelvetia canaliculata); Heartsease (Viola tricolor); Periwinkle of Madagascar (Vinca rosea); Peony; Psyllium, also known as ispaghula (Plantago ovata); Purslane (Portulaca oleracea); Horsetail (Equisetum arvense); Meadowsweet (Spiraea ulmaria); Rose of France (Rosa gallica); Rose of Damascus; Musky rose; Saffron (Crocus sativus); Officinal saponaria (Saponaria officinalis); Clary Sage flower; Saxifrage (Saxifraga sarmentosa); Scutellaria (Scutellaria baicalensis); Elderberry (Sambucus nigra); Large-leaved lime blossom (Tilia platyphyllos); Small-leaved lime blossom (Tilia cordata); Sunflower (Helianthus annuus); Tuberose (Polianthes tuberosa); Valerian (Valeriana officinalis); Bourbon vanilla blossom; Brazilian vanilla blossom (Vanilla planifolia); Lemon verbena (Aloysia citrodora); Violet; Ylang-ylang (Cananga odorata); Hedera helix; Alchillea millefolium; Officinal symphytum; Populus nigra; Salvia officinalis; Plantago major; Arctium lappa; Nigritella rubra; Prunus spinosa; Seabuckthorn blossom

or a mixture of these flowers,
or the following algae: Ascophyllum nodusum; Laminaria; Fucus vesiculosum; Chondrus crispus; Lithotamnium calcareum; Absidinum helminthocorton; Diginea simplex; sea fennel or a mixture of these algae.

In a particular embodiment, the invention relates to the said use in which the said set of flowers comprises or consists of violets or roses or a mixture of these flowers.

In a particular embodiment, the invention relates to the said composition in which the said set of flowers comprises or consists of violets or roses, in particular of roses of Damascus, or a mixture of these flowers.

In a particular embodiment, the invention relates to said composition wherein said flowers or said plants of the set of flowers or plants of the said composition can be fresh flowers or fresh plants, dried flowers or dried plants or a mixture of these flowers or plants.

In another particular embodiment, the invention relates to the said cosmetic composition in which the said vegetable oil is selected between: sweet almond oil, baobab oil, camelina oil, Inca Inchi oil, jojoba oil, hazelnut oil, calophyll oil, andiroba oil, raspberry seed oil, argan oil, borage oil, lemon oil, hemp oil, prickly pear oil, wheat germ oil, cashew nut oil, strawberry seed oil, rose hip oil, avocado oil, seabuckthorn oil, evening primrose oil, apricot kernel oil, macadamia oil, inophyllous calophyll oil, black cumin oil, neem oil, sesame oil, olive oil, monoi oil, papaya oil, safflower oil, rice bran oil, pumpkin seed oil, bellise oil, babassu oil, calendula oil, camellia oil, pequi oil, pracaxi oil, rosehip oil, sunflower oil or copaiba oil or a mixture of these oils.

In yet another particular embodiment, the invention relates to the said cosmetic composition in which the weight ratio between the said vineyard tears and the said vegetable oil of the said composition varies from 0.08 to 16.

This ratio may especially be 0.10; 0.20; 0.30; 0.40; 0.50; 0.6; 0.7; 0.8; 0.9; 1; 1.5; 2; 2.5; 3; 3.5; 4; 4.5; 5; 5.5; 6; 6.5; 7; 7.5; 8; 8.5; 9; 9.5; 10; 10.5; 11; 11.5; 12; 12.5; 13; 13.5; 14; 14.5; 15; 15.5 or 16.

In another particular embodiment, the invention relates to the said composition wherein the said mixture comprises of:
62% of vineyard tears in which violets were initially soaked, and
22% of apricot kernel oil in which said violets were dipped after having been initially soaked in the said vineyard tears,
   or
52% of vineyard tears in which violets were initially soaked, and
31% of apricot kernel oil in which said violets were dipped after having been initially soaked in the said vineyard tears,
   or
52% of vineyard tears in which violets were initially soaked, and
32% of apricot kernel oil in which said violets were dipped after having been initially soaked in the said vineyard tears,
   or
51% of vineyard tears in which violets were initially soaked, and
31% of apricot kernel oil in which said violets were dipped after having been initially soaked in the said vineyard tears,
   or
49% of vineyard tears in which violets were initially soaked, and
40% of apricot kernel oil in which said violets were dipped after having been initially soaked in the said vineyard tears,
   or
46% of vineyard tears in which violets were initially soaked, and
36% of apricot kernel oil in which said violets were dipped after having been initially soaked in the said vineyard tears,
   or
45% of vineyard tears in which violets were initially soaked, and
35% of apricot kernel oil in which said violets were dipped after having been initially soaked in the said vineyard tears,
   or
10% of vineyard tears in which violets were initially soaked, and
10% of apricot kernel oil in which said violets were dipped after having been initially soaked in the said vineyard tears,
   or
20% of vineyard tears in which violets were initially soaked, and
6% of apricot kernel oil in which said violets were dipped after having been initially soaked in the said vineyard tears,

In yet another particular embodiment, the invention relates to the said cosmetic composition comprising from 5% to 80%, in particular from 10% to 80%, in particular from 15% to 75%, in particular from 30% to 70%, and more specifically from 40% to 60% of vineyard tears compared to the total weight of the composition,

from 5% to 60%, in particular from 10% to 60%, in particular from 15% to 50%, in particular from 25% to 40% and more specifically from 30% to 35% of vegetable oil compared to the total weight of the composition,
and from 2% to 8%, in particular 5% of emulsifying agent compared to the total weight of the composition.

“Emulsifying agent” or “emulsifier” means any compound that facilitates the formation of an emulsion between two immiscible liquids.

According to the invention, the emulsifier is preferably selected between natural emulsifying agents, in particular lecithin, lanolin and in particular beeswax.

Emulsifying agents may be associated with fatty alcohols or fatty acids.

The cosmetic composition may contain all adjuvants and additives commonly used in cosmetics.

The cosmetic composition may contain preservatives including cosmegard or geogard.

The cosmetic composition may contain vitamins, especially vitamin A, B, C, D, E, preferably vitamin E.

The said composition may comprise 5%, 10%, 15%, 20%, 25%, 30%, 35%, 40%, 45%, 50%, 55%, 60%, 65%, 70%, 75% or 80% % of vineyard tears compared to the total weight of the composition.

The said composition may comprise 5%, 10%, 15%, 20%, 25%, 30%, 35%, 40%, 45%, 50%, 55% or 60% of vegetable oil compared to the total weight of the composition.

The said composition may comprise 2%, 3%, 4%, 5%, 6%, 7% or 8% of emulsifying agent compared to the total weight of the composition.

In another particular embodiment, the invention relates to said cosmetic composition in which said vineyard tears comprises of the following elements: benzyl alcohol, butanol, butyl acetate, carvacrol, beta-caryophyllenoxide, 1,8-cineol, cuminaldehyde, 1,2-dimethylbenzol, acetic acid, ethyl acetate, beta-famesen, beta-farnesol, hexanol, 1-hexen-3-ol, hexyl acetate, camphor, myristic acid, octanol, palmitic acid, methyl ester, palmitic acid, alpha-pinen, propanol, sabinene, stearic acid, alpha-terpineol, terpinol, terpinyl acetate, tetradecan, thymol, tridecan, 1,2,3-trimethylbenzol, saponin.

In yet another particular embodiment, the invention relates to the said cosmetic composition in which the said mixture comprises of:
Vineyard tears in which violets or roses were initially soaked and
A vegetable oil in which said violets or said roses have been dipped beforehand, after having been initially soaked in the said vineyard tears and
beeswax.

In another particular embodiment, the invention relates to the said cosmetic composition in which the said mixture comprises of:
from 5% to 80%, in particular from 10% to 80%, in particular from 15% to 75%, and in particular from 30% to 70%, and more specifically from 40% to 60% of vineyard tears in which violets or roses were initially soaked, compared to the total weight of the composition,
from 5% to 60%, in particular from 10% to 60%, in particular from 15% to 50%, and in particular from 25% to 40%, and more specifically from 30% to 35% of a vegetable oil, compared to the weight total composition, the said vegetable oil being selected from: sweet almond oil, baobab oil, camelina oil, Inca Inchi oil, jojoba oil, hazelnut oil, calophyll oil, andiroba oil, raspberry seed oil, argan oil, borage oil, lemon oil, hemp oil, prickly pear oil, wheat germ oil, cashew nut oil, strawberry seed oil, rose hip oil, avocado oil, seabuckthorn oil, evening primrose oil, apricot kernel oil, macadamia oil, inophyllous calophyll oil, black cumin oil, neem oil, sesame oil, olive oil, monoi oil, papaya oil, safflower oil, rice bran oil, pumpkin seed oil, bellise oil, babassu oil, calendula oil, camellia oil, pequi oil, pracaxi oil, rosehip oil, sunflower oil or copaiba oil or a mixture of these oils.
   in which said violets or roses, which were initially soaked in said vineyard tears, were dipped beforehand,
5% of beeswax compared to the total weight of the composition.

In another particular embodiment, the invention relates to the said cosmetic composition in which the said mixture comprises of:
from 5% to 80%, in particular from 10% to 80%, in particular from 15% to 75%, and in particular from 30% to 70%, and more specifically from 40% to 60% of vineyard tears in which violets or roses were initially soaked, compared to the total weight of the composition, said vineyard tears comprising of the following elements: benzyl alcohol, butanol, butyl acetate, carvacrol, beta-caryophyllenoxide, 1,8-cineol, cuminaldehyde, 1,2-dimethylbenzol, acetic acid, ethyl acetate, beta-famesen, beta-farnesol, hexanol, 1-hexen-3-ol, hexyl acetate, camphor, myristic acid, octanol, palmitic acid, methyl ester, palmitic acid, alpha-pinen, propanol, sabinene, stearic acid, alpha-terpineol, terpinol, terpinyl acetate, tetradecan, thymol, tridecan, 1,2,3-trimethylbenzol, saponin.
from 5% to 60%, in particular from 10% to 60%, in particular from 15% to 50%, and in particular from 25% to 40%, and more specifically from 30% to 35% of vegetable oil compared to the total weight of the composition, in which said violets or said roses have been dipped beforehand, after being initially soaked in said vineyard tears, the said vegetable oil is selected between: sweet almond oil, baobab oil, camelina oil, Inca Inchi oil, jojoba oil, hazelnut oil, calophyll oil, andiroba oil, raspberry seed oil, argan oil, borage oil, lemon oil, hemp oil, prickly pear oil, wheat germ oil, cashew nut oil, strawberry seed oil, rose hip oil, avocado oil, seabuckthorn oil, evening primrose oil, apricot kernel oil, macadamia oil, inophyllous calophyll oil, black cumin oil, neem oil, sesame oil, olive oil, monoi oil, papaya oil, safflower oil, rice bran oil, pumpkin seed oil, bellise oil, babassu oil, calendula oil, camellia oil, pequi oil, pracaxi oil, rosehip oil, sunflower oil or copaiba oil or a mixture of these oils.
5% of beeswax compared to the total weight of the composition.

In another particular embodiment, the invention relates to the said cosmetic composition in which the said mixture comprises of:
from 5% to 80%, in particular from 10% to 80%, in particular from 15% to 75%, and in particular from 30% to 70%, and more specifically from 40% to 60% of vineyard tears in which violets or roses were initially soaked, compared to the total weight of the composition, the said vineyard tears comprising of the following elements: benzyl alcohol, butanol, butyl acetate, carvacrol, beta-caryophyllenoxide, 1,8-cineol, cuminaldehyde, 1,2-dimethylbenzol, acetic acid, ethyl acetate, beta-famesen, beta-farnesol, hexanol, 1-hexen-3-ol, hexyl acetate, camphor, myristic acid, octanol, palmitic acid, methyl ester, palmitic acid, alpha-pinen, propanol, sabinene, stearic acid, alpha-terpineol, terpinol, terpinyl acetate, tetradecan, thymol, tridecan, 1,2,3-trimethylbenzol, saponin.
from 5% to 60%, in particular from 10% to 60%, in particular from 15% to 50%, and in particular from 25% to 40%, and more specifically from 30% to 35% of a vegetable oil, compared to the weight total composition, in which violets or roses were initially soaked after having been dipped in the said vineyard tears, the said vegetable oil being selected from: sweet almond oil, baobab oil, camelina oil, Inca Inchi oil, jojoba oil, hazelnut oil, calophyll oil, andiroba oil, raspberry seed oil, argan oil, borage oil, lemon oil, hemp oil, prickly pear oil, wheat germ oil, cashew nut oil, strawberry seed oil, rose hip oil, avocado oil, seabuckthorn oil, evening primrose oil, apricot kernel oil, macadamia oil, inophyllous calophyll oil, black cumin oil, neem oil, sesame oil, olive oil, monoi oil, papaya oil, safflower oil, rice bran oil, pumpkin seed oil, bellise oil, babassu oil, calendula oil, camellia oil, pequi oil, pracaxi oil, rosehip oil, sunflower oil or copaiba oil or a mixture of these oils.
5% of beeswax compared to the total weight of the composition.
from 0.001% to 0.01% of a dye mixture from at least one of the following plants: Hedera helix, Alchillea millefolium, Symphytum officinale, Populus nigra, Salvia officinalis, Plantago major, Arctium lappa, Nigritella rubra, Prunus spinosa.

In another particular embodiment, the invention relates to the said cosmetic composition in which the said mixture comprises of:
from 5% to 80%, in particular from 10% to 80%, in particular from 15% to 75%, and in particular from 30% to 70%, and more specifically from 40% to 60% of vineyard tears in which violets or roses were initially soaked, compared to the total weight of the composition, the said vineyard tears comprising of the following elements: benzyl alcohol, butanol, butyl acetate, carvacrol, beta-caryophyllenoxide, 1,8-cineol, cuminaldehyde, 1,2-dimethylbenzol, acetic acid, ethyl acetate, beta-famesen, beta-farnesol, hexanol, 1-hexen-3-ol, hexyl acetate, camphor, myristic acid, octanol, palmitic acid, methyl ester, palmitic acid, alpha-pinen, propanol, sabinene, stearic acid, alpha-terpineol, terpinol, terpinyl acetate, tetradecan, thymol, tridecan, 1,2,3-trimethylbenzol, saponin.
from 5% to 60%, in particular from 10% to 60%, in particular from 15% to 50%, and in particular from 25% to 40%, and more specifically from 30% to 35% of a vegetable oil, compared to the weight total composition, in which violets or roses were initially soaked after having been dipped in the said vineyard tears, the said vegetable oil being selected from: sweet almond oil, baobab oil, camelina oil, Inca Inchi oil, jojoba oil, hazelnut oil, calophyll oil, andiroba oil, raspberry seed oil, argan oil, borage oil, lemon oil, hemp oil, prickly pear oil, wheat germ oil, cashew nut oil, strawberry seed oil, rose hip oil, avocado oil, seabuckthorn oil, evening primrose oil, apricot kernel oil, macadamia oil, inophyllous calophyll oil, black cumin oil, neem oil, sesame oil, olive oil, monoi oil, papaya oil, safflower oil, rice bran oil, pumpkin seed oil, bellise oil, babassu oil, calendula oil, camellia oil, pequi oil, pracaxi oil, rosehip oil, sunflower oil or copaiba oil or a mixture of these oils.
5% of beeswax compared to the total weight of the composition.
from 0.001% to 0.01% of dried fruit powder from grinded dog rose and/or dried leaf powder from grinded dog rose.

In another particular embodiment, the invention relates to the said cosmetic composition in which the said mixture comprises of:
from 5% to 80%, in particular from 10% to 80%, in particular from 15% to 75%, and in particular from 30% to 70%, and more specifically from 40% to 60% of vineyard tears in which violets or roses were initially soaked, compared to the total weight of the composition, the said vineyard tears comprising of the following elements: benzyl alcohol, butanol, butyl acetate, carvacrol, beta-caryophyllenoxide, 1,8-cineol, cuminaldehyde, 1,2-dimethylbenzol, acetic acid, ethyl acetate, beta-famesen, beta-farnesol, hexanol, 1-hexen-3-ol, hexyl acetate, camphor, myristic acid, octanol, palmitic acid, methyl ester, palmitic acid, alpha-pinen, propanol, sabinene, stearic acid, alpha-terpineol, terpinol, terpinyl acetate, tetradecan, thymol, tridecan, 1,2,3-trimethylbenzol, saponin.
from 5% to 60%, in particular from 10% to 60%, in particular from 15% to 50%, and in particular from 25% to 40%, and more specifically from 30% to 35% of a vegetable oil, compared to the weight total composition, in which violets or roses were initially soaked after having been dipped in the said vineyard tears, the said vegetable oil being selected from: sweet almond oil, baobab oil, camelina oil, Inca Inchi oil, jojoba oil, hazelnut oil, calophyll oil, andiroba oil, raspberry seed oil, argan oil, borage oil, lemon oil, hemp oil, prickly pear oil, wheat germ oil, cashew nut oil, strawberry seed oil, rose hip oil, avocado oil, seabuckthorn oil, evening primrose oil, apricot kernel oil, macadamia oil, inophyllous calophyll oil, black cumin oil, neem oil, sesame oil, olive oil, monoi oil, papaya oil, safflower oil, rice bran oil, pumpkin seed oil, bellise oil, babassu oil, calendula oil, camellia oil, pequi oil, pracaxi oil, rosehip oil, sunflower oil or copaiba oil or a mixture of these oils.
5% of beeswax compared to the total weight of the composition.
from 0.001% to 0.01% of a dye mixture from at least one of the following plants: Hedera helix, Alchillea millefolium, Symphytum officinale, Populus nigra, Salvia officinalis, Plantago major, Arctium lappa, Nigritella rubra, Prunus spinosa.
from 0.001% to 0.01% of dried fruit powder from grinded dog rose and/or dried leaf powder from grinded dog rose.

In yet another particular embodiment, the invention relates to the said composition wherein the said mixture comprises of:
62% of vineyard tears in which roses were initially soaked, and
22% of apricot kernel oil in which said roses were dipped after having been initially soaked in the said vineyard tears,
   or
52% of vineyard tears in which roses were initially soaked, and
31% of apricot kernel oil in which said roses were dipped after having been initially soaked in the said vineyard tears,
   or
52% of vineyard tears in which roses were initially soaked, and
32% of apricot kernel oil in which said roses were dipped after having been initially soaked in the said vineyard tears,
   or
51% of vineyard tears in which roses were initially soaked, and
31% of apricot kernel oil in which said roses were dipped after having been initially soaked in the said vineyard tears,
   or
49% of vineyard tears in which roses were initially soaked, and
40% of apricot kernel oil in which said roses were dipped after having been initially soaked in the said vineyard tears,
   or
46% of vineyard tears in which roses were initially soaked, and
36% of apricot kernel oil in which said roses were dipped after having been initially soaked in the said vineyard tears,
   or
45% of vineyard tears in which roses were initially soaked, and
35% of apricot kernel oil in which said roses were dipped after having been initially soaked in the said vineyard tears,
   or
10% of vineyard tears in which roses were initially soaked, and
10% of apricot kernel oil in which said roses were dipped after having been initially soaked in the said vineyard tears,
   or
20% of vineyard tears in which roses were initially soaked, and
6% of apricot kernel oil in which said roses were dipped after having been initially soaked in the said vineyard tears,
   and 5% of emulsifier, in particular beeswax.
   said vineyard tears comprising of the following elements: benzyl alcohol, butanol, butyl acetate, carvacrol, beta-caryophyllenoxide, 1,8-cineol, cuminaldehyde, 1,2-dimethylbenzol, acetic acid, ethyl acetate, beta-famesen, beta-farnesol, hexanol, 1-hexen-3-ol, hexyl acetate, camphor, myristic acid, octanol, palmitic acid, methyl ester, palmitic acid, alpha-pinen, propanol, sabinene, stearic acid, alpha-terpineol, terpinol, terpinyl acetate, tetradecan, thymol, tridecan, 1,2,3-trimethylbenzol, saponin.

In another embodiment, the cosmetic composition of the invention comprises of camel milk and/or sterilized camel urine, or mare milk or donkey milk or sheep milk or goat milk.

In another particular embodiment, the invention relates to a process for preparing a cosmetic composition based on vineyard tears and vegetable oil comprising of the following steps:

(i) immersing a set of flowers or plants or algae in vineyard tears;
(ii) immersing the said set of flowers or plants or algae obtained in step (i) in a vegetable oil;
(iii) grinding the flowers or plants or algae of said set of flowers or plants or algae. Step (iii) being optional or carried out following step (i) or following step (ii).

In another particular embodiment, the invention relates to a process for preparing a cosmetic composition based on vineyard tears and a vegetable oil comprising of the following steps:

(i) immersing a set of flowers or plants or algae in vineyard tears;
(ii) grinding flowers or plants or algae or algae of the said set of flowers or plants or algae previously soaked in vineyard tears, and obtaining a liquid extract;
(iii) mixing the said liquid extract obtained in step (ii) with the said vegetable oil.

In another particular embodiment, the invention relates to a process for preparing a cosmetic composition based on vineyard tears and a vegetable oil comprising of the following steps:

(i) immersing a set of flowers or plants or algae in vineyard tears;
(ii) immersing the said set of flowers or plants or algae obtained in step (i) in a vegetable oil;
(iii) grinding flowers or plants or algae or algae of the said set of flowers or plants obtained in step (ii), and obtaining an oily extract.

In another particular embodiment, the invention relates to a process for preparing a cosmetic composition based on vineyard tears and a vegetable oil comprising of the following steps:

(i) immersing a set of flowers or plants in vineyard tears;
(ii) immersing the said set of flowers or plants obtained in step (i) in a vegetable oil;
(iii) mixing the vineyard tears obtained in step (i) with the vegetable oil obtained in step (ii).

### FIGURES

FIG. 1: UV Chromatogram: HPLC profile of a sample of vineyard tears. Detector: PAD measuring range: from 190 to 700 nm.

FIG. 2: MS Chromatogram: HPLC profile of a sample of vineyard tears. MS detector measuring range from 100 to 1200 m/z. These measurements were performed with UHPLC Acquity H-Class, PDA, ELSD, MS.

FIG. 3: Photographs of the face of a 55 years old female subject before the application of the cosmetic composition (left-hand photograph) and after the application of the cosmetic composition (right-hand photograph).

FIG. 4: Photographs of the face of a 56 years old female subject before the application of the cosmetic composition (left-hand photograph) and after the application of the cosmetic composition (right-hand photograph).

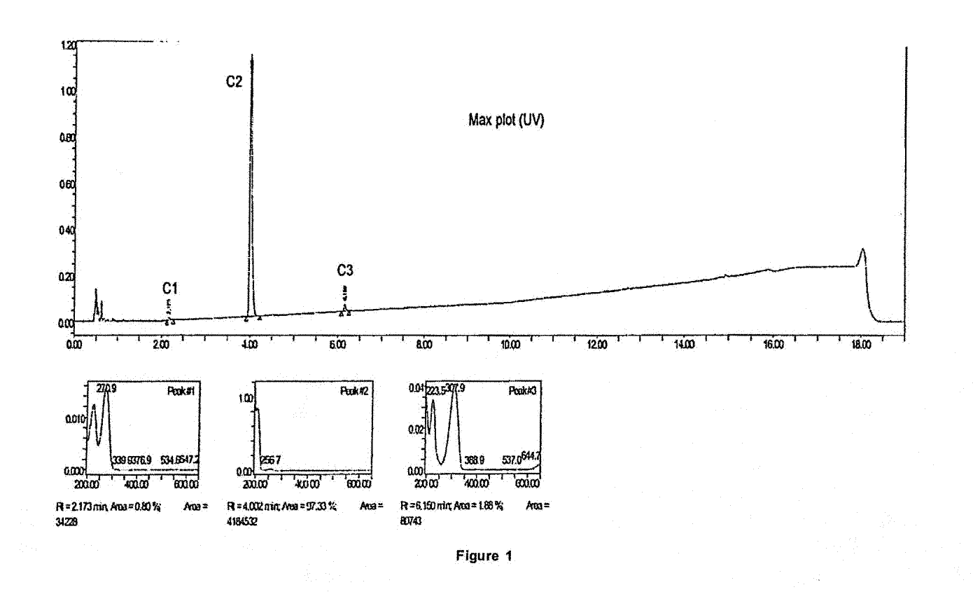

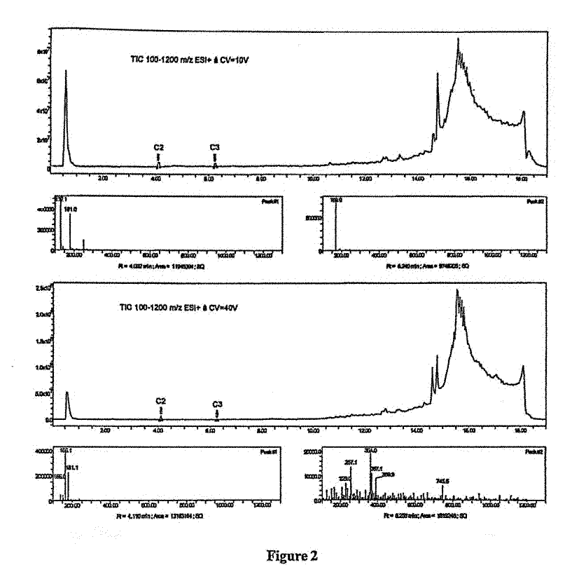

TABLE 1
Summary table of the results obtained

| Com- | Tr | UV | [M + | MS^{z} | Attempt to | Confirmed |
|---|---|---|---|---|---|---|
| pound | (UV) | (λmax) | H]+ | (m/z) | identify | structure |
| C1 | 2.17 | 220, | — | — | No | — |
| | | 271 nm | | | information | |
| C1 | 4.00 | 200, | | 181, 166, | — | — |
| | | 257 nm | | 132 | | |
| C3 | 6.15 | 223, | 169 | 6 | Phenolic | — |
| | | 308 nm | | | compound | |

Three compounds have been highlighted in a sample of vineyard tears. The first (C1) is present in small quantities and has no specific nature enabling us to find out more information about its structure. Compound 2 (C2) is present in significant amounts but does not appear to be a specific feature of the plant. Compound 3 (C3) seems to be a specific feature of the plant. Its UV spectrum shows an important electron delocalization that seems to be linked to the family of phenolic compounds. The mass of C3 (168 g/mol) is related to a polar molecule according to its ratio, size/retention time which corroborates with the affiliation to the phenolic family.

### EXAMPLES OF COSMETIC COMPOSITION

### Example 1

Cosmetic composition for a day cream: “L'Ame de vie—Violettes”

### 1/A

Precious violet oil: apricot kernel oil in which vineyard-tears-soaked violets have been dipped—32%

### A—Beeswax 5%

B—Precious violet serum: vineyard tears in which violets have been soaked beforehand—52%
C—Royal jelly 10%

### C—Collagen 1%

C—Hyaluronic acid 1%

### C—Cosmegard or Geogard 0.6%

### C—Vitamin E 0.4%

### 2/A

Precious violet oil: apricot kernel oil in which vineyard-tears-soaked violets have been dipped—20-60%

### A—Beeswax 2-8%

B—Precious violet serum: vineyard tears in which violets have been soaked beforehand 30-70%
C—Royal jelly 7-14%

### C—Collagen 0.1-4%

### C—0.1-4% Hyaluronic Acid

### C—Cosmegard or Geogard 0.1-3%

### C—Vitamin E 0.1-3%

In the formulations indicated above, dragon blood can be added at a rate of 0.01 to 0.01%

### 3/Implementation

The elements of the phase A are collected in the same container. The elements of phase B are collected in another container. These two phases are then placed in a water bath for a few minutes until the temperature reaches 70° C. Once out of the water bath, the phase B must be transferred to phase A without stopping stirring energetically until the cream whitens, homogenizes and thickens. Then the container containing these two phases should be placed on a little bit of cold water while keeping on stirring energetically for a few minutes until obtaining a very smooth emulsion. Then add the elements of the phase C and mix everything together. The pH of this preparation is about 6-6.5.

The same procedure is applied in Examples 2 to 16.

### Example 2

Cosmetic composition for a night cream: “L'Ame de vie—Violettes”

### 1/A

Precious violet oil: apricot kernel oil in which vineyard-tears-soaked violets have been dipped—36%

### A—Beeswax 5%

B—Precious violet serum: vineyard tears in which violets have been soaked beforehand e—46%
C—Royal jelly 10%

### C—Collagen 1%

### C—Hyaluronic Acid 1%

### C—Cosmegard or Geogard 0.6%

### C—Vitamin E 0.4%

### II/

A—Precious violet oil: apricot kernel oil in which vineyard-tears-soaked violets have been dipped—20-60%

### A—Beeswax 2-8%

B—Precious violet serum: vineyard tears in which violets have been soaked beforehand 25-75%
C—Royal jelly 17-14%

### C—Collagen 0.1-4%

### C—0.1-4% Hyaluronic Acid

### C—Cosmegard or Geogard 0.1-3%

### C—Vitamin E 0.1-3%

### Example 3

### I/

Cosmetic composition for a day cream “L'Ame de vie—Roses de Damas”

A—Precious rose of Damascus oil: apricot kernel oil in which vineyard-tears-soaked roses of Damascus have been dipped—32%

### A—Beeswax 5%

B—Precious rose of Damascus serum: vineyard tears in which roses of Damascus have been soaked beforehand—52%
C—Royal jelly 10%

### C—Collagen 1%

C—Hyaluronic acid 1%

### C—Cosmegard or Geogard 0.6%

### C—Vitamin E 0.4%

### II/

A—Precious rose of Damascus oil: apricot kernel oil in which vineyard-tears-soaked roses of Damascus have been dipped—20-60%

### A—Beeswax 2-8%

B—Precious rose of Damascus serum: vineyard tears in which roses of Damascus have been soaked beforehand—30-70%
C—Royal jelly 7-14%

### C—Collagen 0.1-4%

### C—Hyaluronic Acid 0.1-4%

### C—Cosmegard or Geogard 0.1-3%

### C—Vitamin E 0.1-3%

### Example 4

### I/

Cosmetic composition for a night cream “L'Ame de vie—Roses de Damas”

A—Precious rose of Damascus oil: apricot kernel oil in which vineyard-tears-soaked roses of Damascus have been dipped—36%

### A—Beeswax 5%

B—Precious rose of Damascus serum: vineyard tears in which roses of Damascus have been soaked beforehand—46%
C—Royal jelly 10%

### C—Collagen 1%

### C—Hyaluronic Acid 1%

### C—Cosmegard or Geogard 0.6%

### C—Vitamin E 0.4%

### II/

A—Precious rose of Damascus oil: apricot kernel oil in which vineyard-tears-soaked roses of Damascus have been dipped—20-60%

### A—Beeswax 2-8%

B—Precious rose of Damascus serum: vineyard tears in which roses of Damascus have been soaked beforehand—25-75%
C—Royal jelly 17-14%

### C—Collagen 0.1-4%

### C—Hyaluronic Acid 0.1-4%

### C—Cosmegard or Geogard 0.1-3%

### C—Vitamin E 0.1-3%

### Example 5

### I/

Cosmetic composition for a day cream “Force de vie—Violettes”

A—Precious violet oil: apricot kernel oil in which vineyard-tears-soaked violets have been dipped—31%

### A—Beeswax 5%

B—Precious violet serum: vineyard tears in which violets have been soaked beforehand—52%
C—Royal jelly 10%

### C—Collagen 1%

### C—Hyaluronic Acid 1%

### C—Cosmegard or Geogard 0.6%

### C—Vitamin E 0.4%

### II/

A—Precious violet oil: apricot kernel oil in which vineyard-tears-soaked violets have been dipped—20-60%

### A—Beeswax 2-8%

B—Precious violet serum: vineyard tears in which violets have been soaked beforehand—30-70%
C—Royal jelly 7-14%

### C—Collagen 0.1-4%

### C—0.1-4% Hyaluronic Acid

### C—Cosmegard or Geogard 0.1-3%

### C—Vitamin E 0.1-3%

### Example 6

Cosmetic composition for a night cream “Force de vie—Violettes”

### I/

A—Precious violet oil: apricot kernel oil in which vineyard-tears-soaked violets have been dipped—36%

### A—Beeswax 5%

B—Precious violet serum: vineyard tears in which violets have been soaked beforehand—46%
C—Royal jelly 10%

### C—Collagen 1%

C—Hyaluronic acid 1%

### C—Cosmegard or Geogard 0.6%

### C—Vitamin E 0.4%

### II/

A—Precious violet oil: apricot kernel oil in which vineyard-tears-soaked violets have been dipped—20-60%

### A—Beeswax 2-8%

B—Precious violet serum: vineyard tears in which violets have been soaked beforehand—25-75%
C—Royal jelly 17-14%

### C—Collagen 0.1-4%

C—Life force 0.1-4%

### C—0.1-4% Hyaluronic Acid

### C—Cosmegard or Geogard 0.1-3%

### C—Vitamin E 0.1-3%

### Example 7

Cosmetic composition for a day cream “Force de vie—Roses de Damas”

### I/

A—Precious rose of Damascus oil: apricot kernel oil in which vineyard-tears-soaked roses of Damascus have been dipped—32%

### A—Beeswax 5%

B—Precious rose of Damascus serum: vineyard tears in which roses of Damascus have been soaked beforehand—52%
C—Royal jelly 10%

### C—Collagen 1%

### C—Hyaluronic Acid 1%

### C—Cosmegard or Geogard 0.6%

### C—Vitamin E 0.4%

### II/

A—Precious rose of Damascus oil: apricot kernel oil in which vineyard-tears-soaked roses of Damascus have been dipped—20-60%

### A—Beeswax 2-8%

B—Precious rose of Damascus serum: vineyard tears in which roses of Damascus have been soaked beforehand—30-70%
C—Royal jelly 7-14%

### C—Collagen 0.1-4%

### C—Hyaluronic Acid 0.1-4%

### C—Cosmegard or Geogard 0.1-3%

### C—Vitamin E 0.1-3%

### Example 8

Cosmetic composition for a night cream “Force de vie—Roses de Damas”

### I/

A—Precious rose of Damascus oil: apricot kernel oil in which vineyard-tears-soaked roses of Damascus have been dipped—36%

### A—Beeswax 5%

B—Precious rose of Damascus serum: vineyard tears in which roses of Damascus have been soaked beforehand—46%
C—Royal jelly 10%

### C—Collagen 1%

### C—Hyaluronic Acid 1%

### C—Cosmegard or Geogard 0.6%

### C—Vitamin E 0.4%

### II/

A—Precious rose of Damascus oil: apricot kernel oil in which vineyard-tears-soaked roses of Damascus have been dipped—20-60%

### A—Beeswax 2-8%

B—Precious rose of Damascus serum: vineyard tears in which roses of Damascus have been soaked beforehand—25-75%
C—Royal jelly 17-14%

### C—Collagen 0.1-4%

### C—Hyaluronic Acid 0.1-4%

### C—Cosmegard or Geogard 0.1-3%

### C—Vitamin E 0.1-3%

### Example 9

### I/

Cosmetic composition for a day cream “L'Ame de vie—Violettes”

A—Precious violet oil: apricot kernel oil in which vineyard-tears-soaked violets have been dipped—31%

### A—Beeswax 5%

B—Precious violet serum: vineyard tears in which violets have been soaked beforehand—51%
C—Royal jelly 10%

### C—Cosmegard or Geogard 0.6%

### C—Vitamin E 0.4%

### II/

A—Precious violet oil: apricot kernel oil in which vineyard-tears-soaked violets have been dipped—20-60%

### A—Beeswax 2-8%

B—Precious violet serum: vineyard tears in which violets have been soaked beforehand—30-70%
C—Royal jelly 7-14%

### C—Cosmegard or Geogard 0.1-3%

### C—Vitamin E 0.1-3%

### Example 10

### I/

Cosmetic composition for a night cream “L'Ame de vie—Violettes”

A—Precious violet oil: apricot kernel oil in which vineyard-tears-soaked violets have been dipped—35%

### A—Beeswax 5%

B—Precious violet serum: vineyard tears in which violets have been soaked beforehand—45%
C—Royal jelly 10%

### C—Cosmegard or Geogard 0.6%

### C—Vitamin E 0.4%

### II/

A—Precious violet oil: apricot kernel oil in which vineyard-tears-soaked violets have been dipped—20-60%

### A—Beeswax 2-8%

B—Precious violet serum: vineyard tears in which violets have been soaked beforehand—25-75%
C—Royal jelly 17-14%

### C—Cosmegard or Geogard 0.1-3%

### C—Vitamin E 0.1-3%

### Example 11

Cosmetic composition for a day cream “L'Ame de vie—Roses de Damas”

A—Precious rose of Damascus oil: apricot kernel oil in which vineyard-tears-soaked roses of Damascus have been dipped—31%

### A—Beeswax 5%

B—Precious rose of Damascus serum: vineyard tears in which roses of Damascus have been soaked beforehand—51%
C—Royal jelly 10%

### C—Cosmegard or Geogard 0.6%

### C—Vitamin E 0.4%

### II/

A—Precious rose of Damascus oil: apricot kernel oil in which vineyard-tears-soaked roses of Damascus have been dipped—20-60%

### A—Beeswax 2-8%

B—Precious rose of Damascus serum: vineyard tears in which roses of Damascus have been soaked beforehand—30-70%
C—Royal jelly 7-14%

### C—Cosmegard or Geogard 0.1-3%

### C—Vitamin E 0.1-3%

### Example 12

Cosmetic composition for a night cream “L'Ame de vie—Roses de Damas”

### I/

A—Precious rose of Damascus oil: apricot kernel oil in which vineyard-tears-soaked roses of Damascus have been dipped—35%

### A—Beeswax 5%

B—Precious rose of Damascus serum: vineyard tears in which roses of Damascus have been soaked beforehand—45%
C—Royal jelly 10%

### C—Cosmegard or Geogard 0.6%

### C—Vitamin E 0.4%

### II/

A—Precious rose of Damascus oil: apricot kernel oil in which vineyard-tears-soaked roses of Damascus have been dipped—20-60%

### A—Beeswax 2-8%

B—Precious rose of Damascus serum: vineyard tears in which roses of Damascus have been soaked beforehand—25-75%
C—Royal jelly 17-14%

### C—Cosmegard or Geogard 0.1-3%

### C—Vitamin E 0.1-3%

### Example 13

Cosmetic composition for a face milk “L'Ame de vie—Violettes”

### I/

A—Precious violet oil: apricot kernel oil in which vineyard-tears-soaked violets have been dipped—22%

### A—Beeswax 5%

B—Precious violet serum: vineyard tears in which violets have been soaked beforehand—62%
C—Royal jelly 10%

### C—Collagen 1%

### C—Hyaluronic Acid 1%

### C—Cosmegard or Geogard 0.6%

### C—Vitamin E 0.4%

### II/

A—Precious violet oil: apricot kernel oil in which vineyard-tears-soaked violets have been dipped—10-50%

### A—Beeswax 2-8%

B—Precious violet serum: vineyard tears in which violets have been soaked beforehand—80%
C—Royal jelly 7-14%

### C—Collagen 0.1-4%

### C—0.1-4% Hyaluronic Acid

### C—Cosmegard or Geogard 0.1-3%

### C—Vitamin E 0.1-3%

### Example 14

Cosmetic composition for a body milk “L'Ame de vie—Violettes”

### I/

A—Precious violet oil: apricot kernel oil in which vineyard-tears-soaked violets have been dipped—22%

### A—Beeswax 5%

B—Precious violet serum: vineyard tears in which violets have been soaked beforehand—62%
C—Royal jelly 10%
C—Essential oil of violets 1%
C—Fragrance of violets 1%

### C—Cosmegard or Geogard 0.6%

### C—Vitamin E 0.4%

### II/

A—Precious violet oil: apricot kernel oil in which vineyard-tears-soaked violets have been dipped—10-50%

### A—Beeswax 2-8%

B—Precious violet serum: vineyard tears in which violets have been soaked beforehand—40-80%
C—Royal jelly 17-14%
C—Violet essential oil 0.1-4%
C—Fragrance of violets 0.1-4%

### C—Cosmegard or Geogard 0.1-3%

### C—Vitamin E 0.1-3%

### Example 15

Cosmetic composition for a face milk “L'Ame de vie—Roses de Damas”

### I/

A—Precious rose of Damascus oil: apricot kernel oil in which vineyard-tears-soaked roses of Damascus have been dipped—22% A—Beeswax 5%
B—Precious rose of Damascus serum: vineyard tears in which roses of Damascus have been soaked beforehand—62%
C—Royal jelly 10%

### C—Collagen 1%

### C—Hyaluronic Acid 1%

### C—Cosmegard or Geogard 0.6%

### C—Vitamin E 0.4%

### II/

A—Precious rose of Damascus oil: apricot kernel oil in which vineyard-tears-soaked roses of Damascus have been dipped—10-50%

### A—Beeswax 2-8%

B—Precious rose of Damascus serum: vineyard tears in which roses of Damascus have been soaked beforehand—40-80%
C—Royal jelly 7-14%

### C—Collagen 0.1-4%

### C—0.1-4% Hyaluronic Acid

### C—Cosmegard or Geogard 0.1-3%

### C—Vitamin E 0.1-3%

### Example 16

Cosmetic composition for a body milk “L'Ame de vie—Roses de Damas”

### I/

A—Precious rose of Damascus oil: apricot kernel oil in which vineyard-tears-soaked roses of Damascus have been dipped—22%

### A—Beeswax 5%

B—Precious rose of Damascus serum: vineyard tears in which roses of Damascus have been soaked beforehand—62%
C—Royal jelly 10%
C—Essential oil of Damascus roses 1%
C—Fragrance of roses 1%

### C—Cosmegard or Geogard 0.6%

### C—Vitamin E 0.4%

### II/

A—Precious rose of Damascus oil: apricot kernel oil in which vineyard-tears-soaked roses of Damascus have been dipped—20-60%

### A—Beeswax 2-8%

B—Precious rose of Damascus serum: vineyard tears in which roses of Damascus have been soaked beforehand—25-75%
C—Royal jelly 17-14%
C—essential oil of Damask roses 0.1-4%
C—Fragrance of roses 0.1-4%

### C—Cosmegard or Geogard 0.1-3%

### C—Vitamin E 0.1-3%

### Example 17

Cosmetic composition based on vine gimlet: The Force of Vine—“The soul of vine”

A—Vine gimlet juice 100%

The vine gimlet, also known as vine tendril, is an organ of the vine branch that, like many climbing plants or creepers, enables it to cling to surfaces before it lignifies.

The fresh yin gimlets are grinded, squeezed and crushed to extract the juice.

### Example 18

Precious violet oil—“L'Ame de vie”

### I/

Precious violet oil: apricot kernel oil in which vineyard-tears-soaked violets have been dipped—99%
Vitamin E 1%

### II/Implementation

1 L of apricot kernel oil is placed in a water bath at 70° C. for 2 hours with 250 g of violets. These flowers were soaked for 12 hours in vineyard tears. Once the oily solution has cooled, the flowers are pressed in order to extract the oil, which will also contain the active substances of the flowers.

### Example 19

Precious Oil of Damask Roses—“The Soul of Life”
Precious rose of Damascus oil: apricot kernel oil in which vineyard-tears-soaked roses of Damascus have been dipped—99%
Vitamin E 1%

### Example 20

Precious violet serum: L'Ame de vie

### I/

Vineyard tears in which violets have been soaked beforehand 99%
Vitamin E 1%

### II/Implementation

250 g of violets are immersed in 1 L of vineyard tears for 12 hours. The flowers are then pressed to extract the vineyard tears with all the active substances of the flowers.

### Example 21

Precious rose of Damascus serum: L'Ame de vie
Vineyard tears in which roses of Damascus have been soaked beforehand 99%
Vitamin E 1%

### Example 22

### I/

A—Precious violet oil: apricot kernel oil in which vineyard-tears-soaked violets have been dipped—32%

### A—Beeswax 5%

B—Precious violet serum: vineyard tears in which violets have been soaked beforehand—52%
C—Royal jelly 10%

### C—Collagen 1%

C—Hyaluronic acid 1%

### C—Cosmegard or Geogard 0.6%

### C—Vitamin E 0.4%

### II/Implementation

250 g of violets are immersed in 1 L of vineyard tears for 12 hours. Then the flowers are taken off from the vineyard tears and introduced in 1 L of apricot kernel oil for 2 hours. The flowers are then grinded. The oily extract from this grinding is then mixed with the elements of the phase C.

### Example 23

The same protocol as presented in Example 22 is applied here but with roses of Damascus.

### Example 24

A—Precious violet oil: apricot kernel oil in which vineyard-tears-soaked violets have been dipped—32%

### A—Beeswax 5%

B—Precious violet serum: vineyard tears in which violets have been soaked beforehand—52%
C—Royal jelly 10%

### C—Collagen 1%

C—Hyaluronic acid 1%

### C—Cosmegard or Geogard 0.6%

### C—Vitamin E 0.4%

### II/Implementation

250 g of violets are immersed in 1 L of vineyard tears for 12 hours. The flowers are then grinded. The aqueous extract from this grinding is then mixed with the elements of the phase A and C.

### Example 25

The same protocol as presented in Example 24 is applied here but with roses of Damascus.

### Example 26

Demonstration of the effect of the cosmetic composition

### I/

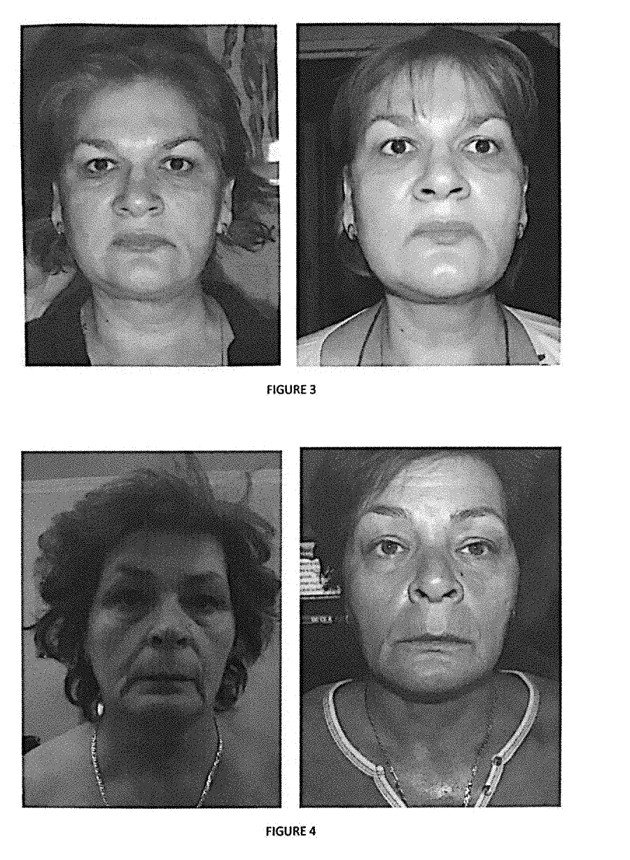

The cosmetic composition according to the invention has been tested on a 55 years old female subject. To determine the effects of this composition the subject was photographed (FIG. 3) before the application of the cosmetic cream (left-hand photograph) and after the application of the cream twice a day for a period of three months (right-handed photograph). It has thus been observed that after the application of the cosmetic composition to the subject's skin, the redness has been greatly reduced, the complexion is much brighter and unified, and the wrinkles are noticeably less marked.

### II/

The same protocol was applied to a 56 years old female subject (FIG. 4).

Once again, after the application of the cosmetic composition, the complexion is brighter, the wrinkles are less marked and the dark circles are much less visible.

1-20. (canceled)

21. Process for preparing a cosmetic composition based on vineyard tears and a vegetable oil comprising of the following steps:
(i) immersing a set of flowers or plants or algae in vineyard tears;
(ii) immersing the said set of flowers or plants or algae obtained in step (i) in a vegetable oil;
(iii) mixing the vineyard tears obtained in step (i) with the vegetable oil obtained in step (ii);
the said composition being free of grape seed polyphenols.

22. Process according to claim 21, comprising an additional step consisting of grinding the flowers or plants or algae of said set of flowers or plants or algae, this step being carried out following step (i) or following step (ii).

23. Process according to claim 21, wherein the flowers or plants or algae of the said set of flowers or plants or algae mentioned above are selected between Agrimony (Agrimonia eupatoria); Alchemilla (Alchemilla vulgaris); Green Anise (Pimpinella anisum) Anthyllis (Anthyllis vulneraria); Armenia white grass (Artemisia herba-alba); Arnica (Arnica montana); Hawthorn (Crataegus oxyacantha); Four o-clock flower (Mirabilis jalapa); Cornflower (Centaurea cyanus); Buddleia; Calendula (Calendula officinalis); Camellia (Camellia sinensis); Chamomile (Anthemis nobilis); Centella (Centella asiatica); Echinacea (Echinacea purpurea); True rosehip; Eleutherococcus; Frangipani (Plumeria alba); Passion fruit flowers; Scotch broom (Cytisus scoparius); Juniper (Juniperus communis); Phenicia juniper; Gentian (Gentiana lutea); Geranium rosat; Pelargonium rosat group; Great comfrey (Symphytum officinale); Marshmallow (Althaea officinalis); Hamamelis (Hamamelis virginiana); Hibiscus; Himanthalia, also known as sea spaghetti (Himanthalia elongata); Immortal; Iris (Iris florentina); Himalayan Jasmine (Nyctantes arbor-tristis); Officinal jasmine, also known as common jasmine (Jasminum officinale), Sambac jasmine, also known as jasmine of Arabia; Daffodil; White cutthroat or white nettle (Lamium album); Laurel; Lavandin; White limnanthes, also known as meadowfoam (Limnanthes alba); white or purple lilac; Lotus; White lupine (Lipinus albus); Lily; Marjoram (Origanum majorana); Horse Chestnut (Aesculus hippocastanus); Mallow or Great Mallow (Malva sylvestris); St. John's wort (Hypericum perforatum); Mimosa (Mimosa tenuiflora); Moringa (Moringa oleifera); Lily of the Valley; Narcissus; White water lily (Nymphea alba); Orange tree (Citrus amara) blossom or fruit pulp; Blue orchid (Dendrobium phalaenopsis); White orchid; Palmarosa (Cymbopogon martini); Peach blossom (Prunus persica); Pelargonium with a strong odor (Pelargonium graveolens); Pelargonium with flowering head (Pelargonium capitatum); Apple blossom; Plum blossom; Pelvetia (Pelvetia canaliculata); Heartsease (Viola tricolor); Periwinkle of Madagascar (Vinca rosea); Peony; Psyllium, also known as ispaghula (Plantago ovata); Purslane (Portulaca oleracea); Horsetail (Equisetum arvense); Meadowsweet (Spiraea ulmaria); Rose of France (Rosa gallica); Rose of Damascus; Musky rose; Saffron (Crocus sativus); Officinal saponaria (Saponaria officinalis); Clary Sage flower Saxifrage (Saxifraga sarmentosa); Scutellaria (Scutellaria baicalensis); Elderberry (Sambucus nigra); Large-leaved lime blossom (Tilia platyphyllos); Small-leaved lime blossom (Tilia cordata); Sunflower (Helianthus annuus); Tuberose (Polianthes tuberosa); Valerian (Valeriana officinalis); Bourbon vanilla blossom; Brazilian vanilla blossom (Vanilla planifolia); Lemon verbena (Aloysia citrodora); Violet; Ylang-ylang (Cananga odorata); Hedera helix; Alchillea millefolium; Officinal symphytum; Populus nigra; Salvia officinalis; Plantago major; Arctium lappa; Nigritella rubra; Prunus spinosa; Seabuckthorn blossom, or a mixture of these flowers,
or the following algae: Ascophyllum nodusum; Laminaria Fucus vesiculosum; Chondrus crispus; Lithotamnium calcareum; Absidinum helminthocorton; Diginea simplex; sea fennel or a mixture of these algae,

24. Process according to claim 21, wherein the said vegetable oil is selected from: sweet almond oil, baobab oil, camelina oil, Inca Inchi oil, jojoba oil, hazelnut oil, calophyll oil, andiroba oil, raspberry seed oil, argan oil, borage oil, lemon oil, hemp oil, prickly pear oil, wheat germ oil, cashew nut oil, strawberry seed oil, rose hip oil, avocado oil, seabuckthorn oil, evening primrose oil, apricot kernel oil, macadamia oil, inophyllous calophyll oil, black cumin oil, neem oil, sesame oil, olive oil, monoi oil, papaya oil, safflower oil, rice bran oil, pumpkin seed oil, bellise oil, babassu oil, calendula oil, camellia oil, pequi oil, pracaxi oil, rosehip oil, sunflower oil or copaiba oil or a mixture of these oils.

25. Process according to claim 21, wherein the said vegetable oil is selected from: sweet almond oil, baobab oil, camelina oil, Inca Inchi oil, jojoba oil, hazelnut oil, calophyll oil, andiroba oil, raspberry seed oil, argan oil, borage oil, lemon oil, hemp oil, prickly pear oil, wheat germ oil, cashew nut oil, strawberry seed oil, rose hip oil, avocado oil, seabuckthorn oil, evening primrose oil, apricot kernel oil, macadamia oil, inophyllous calophyll oil, black cumin oil, neem oil, sesame oil, olive oil, monoi oil, papaya oil, safflower oil, rice bran oil, pumpkin seed oil, bellise oil, babassu oil, calendula oil, camellia oil, pequi oil, pracaxi oil, rosehip oil, sunflower oil or copaiba oil or a mixture of these oils,
and the said set of flowers comprises or is constituted of violets or roses, more specifically roses of Damascus, or a mixture of these flowers.

26. Process according to claim 21, wherein the weight ratio between the said vineyard tears and the said vegetable oil ranges from 0.08 to 16.

27. Cosmetic composition comprising of a mixture of:
vineyard tears in which a set of flowers or plants or algae was initially dipped and,
a vegetable oil in which the said set of flowers or plants or algae was immersed after having been soaked in said vineyard tears,
the said composition being free of grape seed polyphenols.

28. Cosmetic composition according to claim 27, wherein the said set of flowers or plants or algae is grinded after having been soaked in said vineyard tears or after having been soaked in said vegetable oil

29. Cosmetic composition according to claim 27, wherein the flowers or plants or algae of the said set of flowers or plants or algae are selected between: Agrimony (Agrimonia eupatoria); Alchemilla (Alchemilla vulgaris); Green Anise (Pimpinella anisum) Anthyllis (Anthyllis vulneraria); Armenia white grass (Artemisia herba-alba); Arnica (Arnica montana); Hawthorn (Crataegus oxyacantha) Four o-clock flower (Mirabilis jalapa); Cornflower (Centaurea cyanus); Buddleia Calendula (Calendula officinalis); Camellia (Camellia sinensis); Chamomile (Anthemis nobilis); Centella (Centella asiatica); Echinacea (Echinacea purpurea); True rosehip; Eleutherococcus; Frangipani (Plumeria alba); Passion fruit flowers; Scotch broom (Cytisus scoparius); Juniper (Juniperus communis); Phenicia juniper; Gentian (Gentiana lutea); Geranium rosat; Pelargonium rosat group; Great comfrey (Symphytum officinale) Marshmallow (Althaea officinalis); Hamamelis (Hamamelis virginiana); Hibiscus; Himanthalia, also known as sea spaghetti (Himanthalia elongata); Immortal; Iris (Iris florentina); Himalayan Jasmine (Nyctantes arbor-tristis); Officinal jasmine, also known as common jasmine (Jasminum officinale), Sambac jasmine, also known as jasmine of Arabia; Daffodil; White cutthroat or white nettle (Lamium album); Laurel; Lavandin; White limnanthes, also known as meadowfoam (Limnanthes alba); white or purple lilac; Lotus; White lupine (Lipinus albus); Lily; Marjoram (Origanum majorana); Horse Chestnut (Aesculus hippocastanus); Mallow or Great Mallow (Malva sylvestris); St. John's wort (Hypericum perforatum); Mimosa (Mimosa tenuiflora); Moringa (Moringa oleifera); Lily of the Valley; Narcissus; White water lily (Nymphea alba); Orange tree (Citrus amara) blossom or fruit pulp; Blue orchid (Dendrobium phalaenopsis); White orchid; Palmarosa (Cymbopogon martini); Peach blossom (Prunus persica); Pelargonium with a strong odor (Pelargonium graveolens); Pelargonium with flowering head (Pelargonium capitatum); Apple blossom; Plum blossom; Pelvetia (Pelvetia canaliculata); Heartsease (Viola tricolor); Periwinkle of Madagascar (Vinca rosea); Peony; Psyllium, also known as ispaghula (Plantago ovata); Purslane (Portulaca oleracea); Horsetail (Equisetum arvense); Meadowsweet (Spiraea ulmaria); Rose of France (Rosa gallica); Rose of Damascus; Musky rose; Saffron (Crocus sativus); Officinal saponaria (Saponaria officinalis); Clary Sage flower Saxifrage (Saxifraga sarmentosa); Scutellaria (Scutellaria baicalensis); Elderberry (Sambucus nigra); Large-leaved lime blossom (Tilia platyphyllos); Small-leaved lime blossom (Tilia cordata); Sunflower (Helianthus annuus); Tuberose (Polianthes tuberosa); Valerian (Valeriana officinalis); Bourbon vanilla blossom; Brazilian vanilla blossom (Vanilla planifolia); Lemon verbena (Aloysia citrodora); Violet; Ylang-ylang (Cananga odorata); Hedera helix; Alchillea millefolium; Officinal symphytum; Populus nigra; Salvia officinalis; Plantago major; Arctium lappa; Nigritella rubra; Prunus spinosa; Seabuckthorn blossom, or a mixture of these flowers,
or the following algae: Ascophyllum nodusum; Laminaria; Fucus vesiculosum; Chondrus crispus; Lithotamnium calcareum; Absidinum helminthocorton; Diginea simplex; sea fennel or a mixture of these algae,

30. Cosmetic composition according to claim 27, wherein the said vegetable oil is selected from: sweet almond oil, baobab oil, camelina oil, Inca Inchi oil, jojoba oil, hazelnut oil, calophyll oil, andiroba oil, raspberry seed oil, argan oil, borage oil, lemon oil, hemp oil, prickly pear oil, wheat germ oil, cashew nut oil, strawberry seed oil, rose hip oil, avocado oil, seabuckthorn oil, evening primrose oil, apricot kernel oil, macadamia oil, inophyllous calophyll oil, black cumin oil, neem oil, sesame oil, olive oil, monoi oil, papaya oil, safflower oil, rice bran oil, pumpkin seed oil, bellise oil, babassu oil, calendula oil, camellia oil, pequi oil, pracaxi oil, rosehip oil, sunflower oil or copaiba oil or a mixture of these oils.

31. Cosmetic composition according to claim 27, wherein the said vegetable oil is selected from: sweet almond oil, baobab oil, camelina oil, Inca Inchi oil, jojoba oil, hazelnut oil, calophyll oil, andiroba oil, raspberry seed oil, argan oil, borage oil, lemon oil, hemp oil, prickly pear oil, wheat germ oil, cashew nut oil, strawberry seed oil, rose hip oil, avocado oil, seabuckthorn oil, evening primrose oil, apricot kernel oil, macadamia oil, inophyllous calophyll oil, black cumin oil, neem oil, sesame oil, olive oil, monoi oil, papaya oil, safflower oil, rice bran oil, pumpkin seed oil, bellise oil, babassu oil, calendula oil, camellia oil, pequi oil, pracaxi oil, rosehip oil, sunflower oil or copaiba oil or a mixture of these oils,
and the said set of flowers comprises or is constituted of violets or roses, more specifically roses of Damascus, or a mixture of these flowers.

32. Cosmetic composition according to claim 27, wherein said mixture comprises of:
62% of vineyard tears in which violets were initially soaked, and
22% of apricot kernel oil in which said violets were dipped after having been initially soaked in the said vineyard tears,
or
52% of vineyard tears in which violets were initially soaked, and
31% of apricot kernel oil in which said violets were dipped after having been initially soaked in the said vineyard tears,
or
52% of vineyard tears in which violets were initially soaked, and
32% of apricot kernel oil in which said violets were dipped after having been initially soaked in the said vineyard tears,
or
51% of vineyard tears in which violets were initially soaked, and
31% of apricot kernel oil in which said violets were dipped after having been initially soaked in the said vineyard tears,
or
49% of vineyard tears in which violets were initially soaked, and
40% of apricot kernel oil in which said violets were dipped after having been initially soaked in the said vineyard tears,
or
46% of vineyard tears in which violets were initially soaked, and
36% of apricot kernel oil in which said violets were dipped after having been initially soaked in the said vineyard tears,
or
45% of vineyard tears in which violets were initially soaked, and
35% of apricot kernel oil in which said violets were dipped after having been initially soaked in the said vineyard tears,
or
10% of vineyard tears in which violets were initially soaked, and
10% of apricot kernel oil in which said violets were dipped after having been initially soaked in the said vineyard tears,
or
20% of vineyard tears in which violets were initially soaked, and
6% of apricot kernel oil in which said violets were dipped after having been initially soaked in the said vineyard tears,
the said percentages being expressed compared to the total weight of the composition.

33. Cosmetic composition according to claim 27,
comprising of:
from 5% to 80%, in particular from 10% to 80%, in particular from 15% to 75%, in particular from 30% to 70%, and more specifically from 40% to 60% of vineyard tears compared to the total weight of the composition,
from 5% to 60%, in particular from 10% to 60%, in particular from 15% to 50%, in particular from 25% to 40% and more specifically from 30% to 35% of vegetable oil compared to the total weight of the composition,
and from 2% to 8%, more specifically 5% of emulsifying agent compared to the total weight of the composition.

34. Cosmetic composition according to claim 27, wherein the said vineyard tears comprise of the following elements: benzyl alcohol, butanol, butyl acetate, carvacrol, beta-caryophyllenoxide, 1,8-cineol, cuminaldehyde, 1,2-dimethylbenzol, acetic acid, ethyl acetate, beta-famesen, beta-famesol, hexanol, 1-hexen-3-ol, hexyl acetate, camphor, myristic acid, octanol, palmitic acid, methyl ester, palmitic acid, alpha-pinen, propanol, sabinene, stearic acid, alpha-terpineol, terpinol, terpinyl acetate, tetradecan, thymol, tridecan, 1,2,3-trimethylbenzol, saponin.

35. Cosmetic composition according to claim 27, wherein the said mixture comprises of:
vineyard tears in which violets or roses were initially dipped and,
a vegetable oil in which the said violets or the said roses were immersed after having been soaked in the said vineyard tears,
beeswax

36. Cosmetic composition according to claim 27, in which the said mixture comprises of:
from 5% to 80%, in particular from 10% to 80%, in particular from 15% to 75%, in particular from 30% to 70%, and more specifically from 40% to 60% of vineyard tears in which violets or roses initially soaked compared to the total weight of the composition,
from 5% to 60%, in particular from 10% to 60%, in particular from 15% to 50%, in particular from 25% to 40% and more specifically from 30% to 35% of a vegetable oil compared to the weight total composition, said vegetable oil being selected between: sweet almond oil, baobab oil, camelina oil, Inca Inchi oil, jojoba oil, hazelnut oil, calophyll oil, andiroba oil, raspberry seed oil, argan oil, borage oil, lemon oil, hemp oil, prickly pear oil, wheat germ oil, cashew nut oil, strawberry seed oil, rose hip oil, avocado oil, seabuckthorn oil, evening primrose oil, apricot kernel oil, macadamia oil, inophyllous calophyll oil, black cumin oil, neem oil, sesame oil, olive oil, monoi oil, papaya oil, safflower oil, rice bran oil, pumpkin seed oil, bellise oil, babassu oil, calendula oil, camellia oil, pequi oil, pracaxi oil, rosehip oil, sunflower oil or copaiba oil or a mixture of these oils,
in which the said violets or roses, which were initially soaked in the said vineyard tears, were immersed beforehand,
5% of beeswax compared to the total weight of the composition.

37. Cosmetic composition according to claim 27, wherein the said mixture comprises of:
from 5% to 80%, in particular from 10% to 80%, in particular from 15% to 75%, and in particular from 30% to 70%, and more specifically from 40% to 60% of vineyard tears in which violets or roses were initially soaked, compared to the total weight of the composition, the said vineyard tears comprising of the following elements: benzyl alcohol, butanol, butyl acetate, carvacrol, beta-caryophyllenoxide, 1,8-cineol, cuminaldehyde, 1,2-dimethylbenzol, acetic acid, ethyl acetate, beta-famesen, beta-famesol, hexanol, 1-hexen-3-ol, hexyl acetate, camphor, myristic acid, octanol, palmitic acid, methyl ester, palmitic acid, alpha-pinen, propanol, sabinene, stearic acid, alpha-terpineol, terpinol, terpinyl acetate, tetradecan, thymol, tridecan, 1,2,3-trimethylbenzol, saponin;
from 5% to 60%, in particular from 10% to 60%, in particular from 15% to 50%, and in particular from 25% to 40%, and more specifically from 30% to 35% of a vegetable oil, compared to the weight total composition, in which violets or roses were initially soaked after having been dipped in the said vineyard tears, the said vegetable oil being selected from: sweet almond oil, baobab oil, camelina oil, Inca Inchi oil, jojoba oil, hazelnut oil, calophyll oil, andiroba oil, raspberry seed oil, argan oil, borage oil, lemon oil, hemp oil, prickly pear oil, wheat germ oil, cashew nut oil, strawberry seed oil, rose hip oil, avocado oil, seabuckthorn oil, evening primrose oil, apricot kernel oil, macadamia oil, inophyllous calophyll oil, black cumin oil, neem oil, sesame oil, olive oil, monoi oil, papaya oil, safflower oil, rice bran oil, pumpkin seed oil, bellise oil, babassu oil, calendula oil, camellia oil, pequi oil, pracaxi oil, rosehip oil, sunflower oil or copaiba oil or a mixture of these oils;
5% of beeswax compared to the total weight of the composition;
from 0.001% to 0.01% of a dye mixture from at least one of the following plants: Hedera helix, Alchillea millefolium, Symphytum officinale, Populus nigra, Salvia officinalis, Plantago major, Arctium lappa, Nigritella rubra, Prunus spinosa.

38. Cosmetic composition according to claim 27, wherein the said mixture comprises of:
from 5% to 80%, in particular from 10% to 80%, in particular from 15% to 75%, and in particular from 30% to 70%, and more specifically from 40% to 60% of vineyard tears in which violets or roses were initially soaked, compared to the total weight of the composition, the said vineyard tears comprising of the following elements: benzyl alcohol, butanol, butyl acetate, carvacrol, beta-caryophyllenoxide, 1,8-cineol, cuminaldehyde, 1,2-dimethylbenzol, acetic acid, ethyl acetate, beta-famesen, beta-famesol, hexanol, 1-hexen-3-ol, hexyl acetate, camphor, myristic acid, octanol, palmitic acid, methyl ester, palmitic acid, alpha-pinen, propanol, sabinene, stearic acid, alpha-terpineol, terpinol, terpinyl acetate, tetradecan, thymol, tridecan, 1,2,3-trimethylbenzol, saponin;
from 5% to 60%, in particular from 10% to 60%, in particular from 15% to 50%, and in particular from 25% to 40%, and more specifically from 30% to 35% of a vegetable oil, compared to the weight total composition, in which violets or roses were initially soaked after having been dipped in the said vineyard tears, the said vegetable oil being selected from: sweet almond oil, baobab oil, camelina oil, Inca Inchi oil, jojoba oil, hazelnut oil, calophyll oil, andiroba oil, raspberry seed oil, argan oil, borage oil, lemon oil, hemp oil, prickly pear oil, wheat germ oil, cashew nut oil, strawberry seed oil, rose hip oil, avocado oil, seabuckthorn oil, evening primrose oil, apricot kernel oil, macadamia oil, inophyllous calophyll oil, black cumin oil, neem oil, sesame oil, olive oil, monoi oil, papaya oil, safflower oil, rice bran oil, pumpkin seed oil, bellise oil, babassu oil, calendula oil, camellia oil, pequi oil, pracaxi oil, rosehip oil, sunflower oil or copaiba oil or a mixture of these oils;
5% of beeswax compared to the total weight of the composition;
from 0.001% to 0.01% of dried fruit powder from grinded dog rose and/or dried leaf powder from grinded dog rose.

39. Cosmetic composition according to claim 27, wherein the said mixture comprises of:
from 5% to 80%, in particular from 10% to 80%, in particular from 15% to 75%, and in particular from 30% to 70%, and more specifically from 40% to 60% of vineyard tears in which violets or roses were initially soaked, compared to the total weight of the composition, the said vineyard tears comprising of the following elements: benzyl alcohol, butanol, butyl acetate, carvacrol, beta-caryophyllenoxide, 1,8-cineol, cuminaldehyde, 1,2-dimethylbenzol, acetic acid, ethyl acetate, beta-famesen, beta-famesol, hexanol, 1-hexen-3-ol, hexyl acetate, camphor, myristic acid, octanol, palmitic acid, methyl ester, palmitic acid, alpha-pinen, propanol, sabinene, stearic acid, alpha-terpineol, terpinol, terpinyl acetate, tetradecan, thymol, tridecan, 1,2,3-trimethylbenzol, saponin;
from 5% to 60%, in particular from 10% to 60%, in particular from 15% to 50%, and in particular from 25% to 40%, and more specifically from 30% to 35% of a vegetable oil, compared to the weight total composition, in which violets or roses were initially soaked after having been dipped in the said vineyard tears, the said vegetable oil being selected from: sweet almond oil, baobab oil, camelina oil, Inca Inchi oil, jojoba oil, hazelnut oil, calophyll oil, andiroba oil, raspberry seed oil, argan oil, borage oil, lemon oil, hemp oil, prickly pear oil, wheat germ oil, cashew nut oil, strawberry seed oil, rose hip oil, avocado oil, seabuckthorn oil, evening primrose oil, apricot kernel oil, macadamia oil, inophyllous calophyll oil, black cumin oil, neem oil, sesame oil, olive oil, monoi oil, papaya oil, safflower oil, rice bran oil, pumpkin seed oil, bellise oil, babassu oil, calendula oil, camellia oil, pequi oil, pracaxi oil, rosehip oil, sunflower oil or copaiba oil or a mixture of these oils;
5% of beeswax compared to the total weight of the composition;
from 0.001% to 0.01% of a dye mixture from at least one of the following plants: Hedera helix, Alchillea millefolium, Symphytum officinale, Populus nigra, Salvia officinalis, Plantago major, Arctium lappa, Nigritella rubra, Prunus spinosa;
from 0.001% to 0.01% of dried fruit powder from grinded dog rose and/or dried leaf powder from grinded dog rose.

40. Cosmetic composition according to claim 27, wherein the said mixture comprises of camel milk and/or sterilized camel urine, or mare milk, or donkey milk, or sheep milk, or goat milk.
